(19) United States
(12) Patent Application Publication
(10) Pub. No.: US 2025/0248778 A1
(43) Pub. Date: Aug. 7, 2025

(54) SURGICAL ROBOTIC SYSTEM FOR END EFFECTOR SCALING CONTROL

(71) Applicant: Covidien LP, Mansfield, MA (US)

(72) Inventors: Connor D. Roberts, Arvada, CO (US); Haralambos P. Apostolopoulos, Westminster, CO (US); Christopher T. Tschudy, Arvada, CO (US)

(21) Appl. No.: 19/008,098

(22) Filed: Jan. 2, 2025

Related U.S. Application Data
(60) Provisional application No. 63/627,896, filed on Feb. 1, 2024.

(51) Int. Cl. A61B 34/00; A61B 34/37

(52) U.S. Cl. CPC A61B 34/77; A61B 34/25; A61B 34/37; A61B 34/74; A61B 34/76

(57) ABSTRACT

A surgical robotic system includes an instrument and a surgeon console for setting a maximum opening aperture for an end effector of an instrument. The end effector has a first jaw and a second jaw, at least one of which is moveable relative to the other between closed and open configurations. The maximum opening aperture is a variable which limits the open configuration of the first and second jaws. The open and closed configurations of the first and second jaws are controlled by the input controller, which may include a handle controller or a foot pedal. The maximum opening aperture of the first and second jaws may be set on a display screen for receiving user input. The user input may be entered via interaction with a user interface element, such as a user-adjustable graphical representation of the instrument, a dropdown menu, an alphanumeric input box, a slider, etc.

### CROSS-REFERENCE TO RELATED APPLICATIONS

This application claims the benefit of, and priority to, U.S. Provisional Patent Application Ser. No. 63/627,896 filed on Feb. 1, 2024. The entire contents of the foregoing application are incorporated by reference herein.

### BACKGROUND

Surgical robotic systems are currently being used in a variety of medical procedures, including minimally invasive surgical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body. A variety of different types of instruments are used with surgical robotic systems that are designed to perform specific functions.

### SUMMARY

Laparoscopic and robotic surgical procedures utilize different types of instruments with unique end effectors, for example, a gripping end effector may be used to perform fine blunt dissection procedures. Clinicians request the ability to modify certain operational parameters of the instruments, e.g., such that during blunt dissections, the jaws of the grasper do not open to their fully open position, as that may unintentionally tear tissue and cause complications. The present disclosure provides for setting a maximum aperture size of an end effector, which allows a user to set a desired maximum aperture (i.e., jaw opening angle) based on the procedure being performed.

According to one aspect of the present disclosure, a surgical robotic system includes an instrument and a surgeon console. The instrument includes an end effector having a first jaw and a second jaw. The first jaw and/or the second jaw is movable relative to the other of the first jaw or the second jaw between a closed configuration and an open configuration. The surgeon console includes an input controller and a display screen. The input controller receives a first input corresponding to the closed configuration of the first and second jaws and a second input corresponding to the open configuration of the first and second jaws. The display screen displays a graphical user interface for receiving a user input for setting a maximum opening aperture for the open configuration of the first and second jaws.

In another aspect of the present disclosure, the surgical robotic system may further include an instrument drive unit coupled to the instrument.

In yet another aspect of the present disclosure, the instrument drive unit may include a controller and one or more motors for moving the first jaw and/or the second jaw between the closed configuration and the open configuration.

In a further aspect of the present disclosure, the controller may open the first jaw and/or the second jaw up to the maximum opening aperture.

In yet a further aspect of the present disclosure, the graphical user interface may include a user interface element for setting the maximum opening aperture.

In another aspect of the present disclosure, the user interface element may be a user-adjustable graphical representation of the instrument.

In yet another aspect of the present disclosure, the user interface element may be a dropdown menu, an alphanumeric input box, a slider, or a combination thereof.

In a further aspect of the present disclosure, the display screen may include a touch panel for receiving the user input.

In yet a further aspect of the present disclosure, the input controller may be a handle controller or a foot pedal.

In another aspect of the present disclosure, the input controller may include a handle and a paddle. The paddle may be pivotably movable relative to the handle from a closed position corresponding to the closed configuration of the first and second jaws and an open position corresponding to the open configuration of the first and second jaws.

In yet another aspect of the present disclosure, the input controller may further include a trigger moveable relative to the handle from a deactivated position corresponding to the closed configuration of the first and second jaws and an activated position corresponding to the open configuration of the first and second jaws.

In a further aspect of the present disclosure, the input controller may further include a haptic feedback device for generating haptic feedback when the paddle is in the closed position and/or the open position.

### BRIEF DESCRIPTION OF THE DRAWINGS

Various embodiments of the present disclosure are described herein with reference to the drawings, wherein:

FIG. 1 is a perspective view of a surgical robotic system including a control tower, a console, and one or more surgical robotic arms, each disposed on a movable cart according to an embodiment of the present disclosure;

FIG. 2 is a perspective view of a surgical robotic arm of the surgical robotic system of FIG. 1 according to an embodiment of the present disclosure;

FIG. 3 is a perspective view of a movable cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of movable carts of FIG. 1 positioned about a surgical table according to an embodiment of the present disclosure;

FIG. 6 is a perspective view, with parts separated, of an instrument drive unit and a surgical instrument according to an embodiment of the present disclosure;

FIG. 7 is a perspective view of an end effector, according to an embodiment of the present disclosure, for use in the surgical robotic system of FIG. 1;

FIG. 8 shows the end effector in various configurations according to an embodiment of the present disclosure;

FIG. 9 is a perspective view of a handle controller according to an embodiment of the present disclosure; and

FIG. 10 is an exemplary GUI display for an end effector scaling control system according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views.

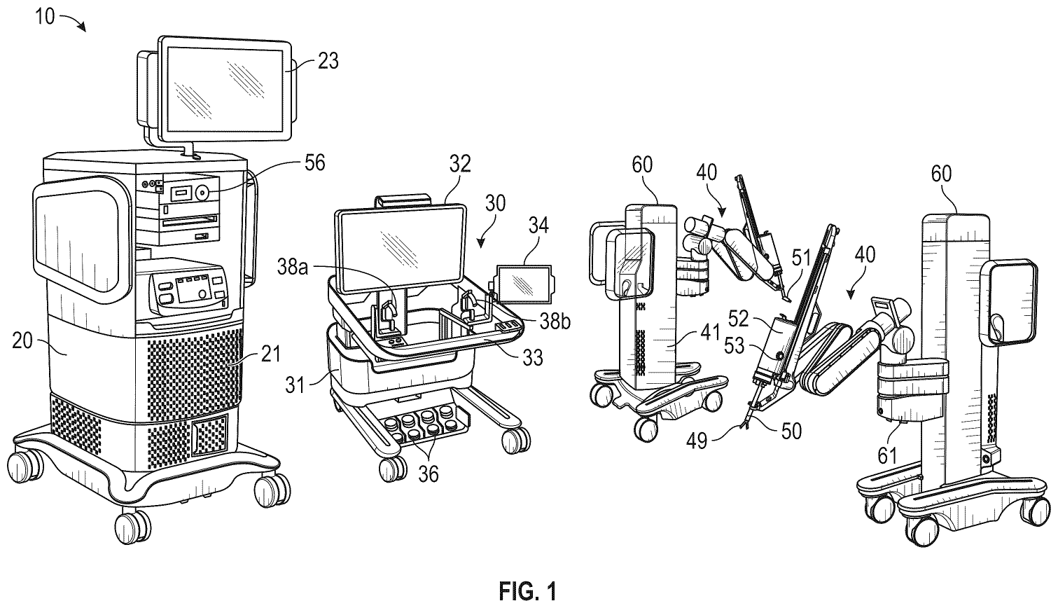

With reference to FIG. 1, a surgical robotic system 10 includes a control tower 20, which is communicatively coupled to all of the components of the surgical robotic system 10 including a surgeon console 30 and one or more movable carts 60. Each of the movable carts 60 includes a robotic arm 40 having an instrument 50 coupled thereto. The robotic arms 40 also couple to the movable carts 60. The surgical robotic system 10 may include any number of movable carts 60 and/or robotic arms 40.

The instrument 50 is configured for use during minimally invasive surgical procedures. In embodiments, the instrument 50 may be configured for open surgical procedures. One of the robotic arms 40 may include an endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 and output the processed video stream.

The surgeon console 30 includes a first display 32, which displays a video feed of the surgical site provided by a camera 51 disposed on the robotic arm 40, and a second display 34, which displays a user interface for controlling the surgical robotic system 10. The first display 32 and the second display 34 may be touchscreens allowing for displaying various graphical user inputs selectable or movable by the user.

The surgeon console 30 also includes a plurality of user interface devices, such as foot pedals 36 and a pair of handle controllers 38a and 38b, which are used by a user to remotely control the robotic arms 40. The surgeon console further includes an armrest 33 used to support clinician's arms while the clinician is operating the handle controllers 38a and 38b.

The control tower 20 can also include a display 23, which may be a touchscreen, and outputs on the graphical user interfaces (GUIs). The control tower 20 also acts as an interface between the surgeon console 30 and one or more of the robotic arms 40. In particular, the control tower 20 is configured to control the robotic arms 40, such as to move the robotic arms 40 and the corresponding instrument 50, based on a set of programmable instructions and/or input commands from the surgeon console 30. In response to the instructions and/or input, the robotic arms 40 and the instrument 50 execute a desired movement sequence in response to input from the foot pedals 36 and the handle controllers 38a and 38b. The surgical robotic system 10 can be configured so that the foot pedals 36 may be used to affect one or more of a wide variety of system functions, such as to enable and lock the handle controllers 38a and 38b, reposition camera movement, and activate/deactivate an electrosurgical instrument. In particular, the foot pedals 36 may be used to perform a clutching action on the handle controllers 38a and 38b. Clutching is initiated by pressing one of the foot pedals 36, which disconnects (i.e., prevents movement inputs from) the handle controllers 38a and/or 38b such that the robotic arm 40 and corresponding instrument 50 or camera 51 are not actuated. This allows the user to reposition the handle controllers 38a and 38b without moving the robotic arm(s) 40 and the instrument 50 and/or camera 51. This is useful when reaching control boundaries of the surgical space, for instance.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency (RF), optical, Wi-Fi, Bluetooth (an open wireless protocol for exchanging data over short distances, using short-length radio waves, from fixed and mobile devices, creating personal area networks (PANs), ZigBee® (a specification for a suite of high-level communication protocols using small, low-power digital radios based on the IEEE 122.15.4-1203 standard for wireless personal area networks (WPANs)).

The computers 21, 31, 41 may include any suitable processor (not shown) connected operably to a memory (not shown), which may include one or more of volatile, non-volatile, magnetic, optical, or electrical media, such as read-only memory (ROM), random access memory (RAM), electrically erasable programmable ROM (EEPROM), non-volatile RAM (NVRAM), or flash memory. The processor may be any suitable processor (e.g., control circuit) adapted to perform the operations, calculations, and/or set of instructions described in the present disclosure, such as a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

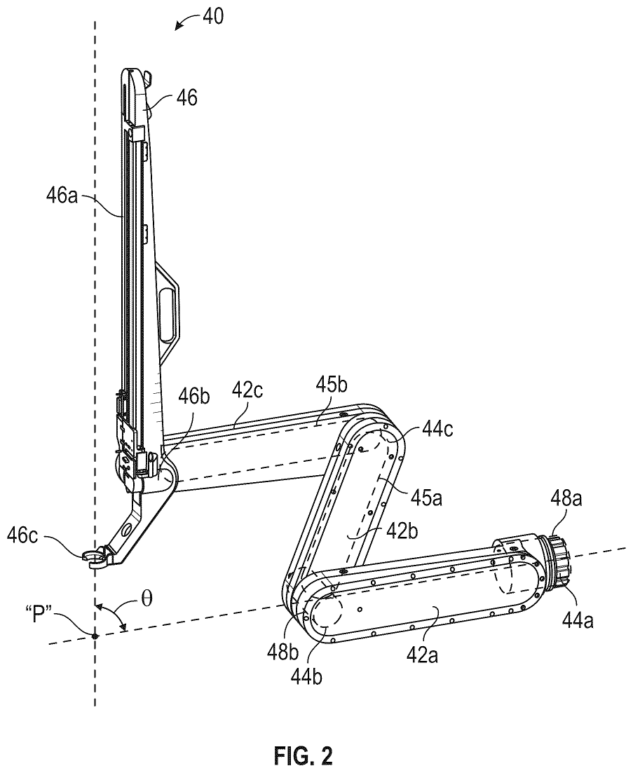

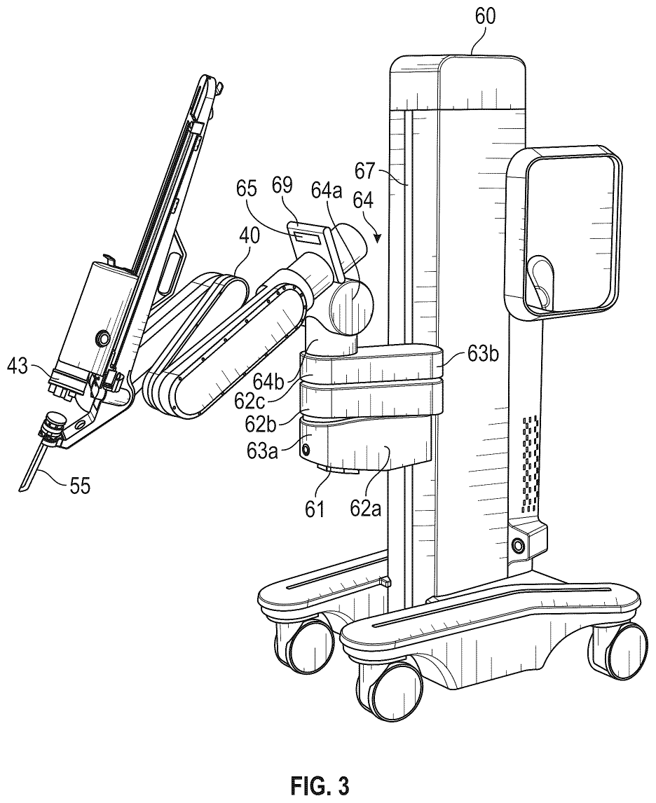

With reference to FIG. 2, each of the robotic arms 40 may include a plurality of links 42a, 42b, 42c, which are interconnected at joints 44b and 44c, respectively. Other configurations of links and joints may be utilized as known by those skilled in the art. The joint 44a is configured to secure the robotic arm 40 to the movable cart 60 and defines a first longitudinal axis. With reference to FIG. 3, the movable cart 60 includes a lift 67 and a setup arm 61, which provides a base for mounting of the robotic arm 40. The lift 67 allows for vertical movement of the setup arm 61 and, thereby, of the robotic arms 40 mounted on the setup arm 61. The movable cart 60 also includes a display 69 for displaying information pertaining to the robotic arm 40. In embodiments, the robotic arms 40 may include any type and/or number of joints.

With further reference to FIG. 3, the setup arm 61 includes a first link 62a, a second link 62b, and a third link 62c, which provide for lateral maneuverability of the robotic arms 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in corresponding lateral planes, which are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis, which is perpendicular to a plane defined by the third link 62c. And the second actuator 64b is rotatable about a second stationary arm axis which is transverse to the first stationary arm axis. The first and second actuators 64a and 64b along with the lift 67 allow for full three-dimensional orientation of the robotic arm 40.

Returning to FIG. 2, the actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include respective actuators 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the instrument 50 and the camera 51 and is configured to move (e.g., rotate) and actuate the instrument 50 and/or the camera 51. IDU 52 transfers actuation forces from its actuators to the instrument 50 to actuate components of an end effector 49 of the instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an access port 55 (FIG. 3) held by the holder 46. The holder 46 also includes a port latch 46c for securing the access port 55 to the holder 46 (FIG. 2).

The IDU 52 is attached to the holder 46, followed by a sterile interface module (SIM) 43 being attached to a distal portion of the IDU 52. The SIM 43 is configured to secure a sterile drape (not shown) to the IDU 52. The instrument 50 is then attached to the SIM 43. The instrument 50 is then inserted through the access port 55 by moving the IDU 52 along the holder 46. The SIM 43 includes a plurality of drive shafts configured to transmit rotation of individual motors of the IDU 52 to instrument 50 (e.g., a surgical instrument) thereby actuating the instrument 50. In addition, the SIM 43 provides a sterile barrier between the instrument 50 and the other components of the robotic arm 40, including the IDU 52.

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

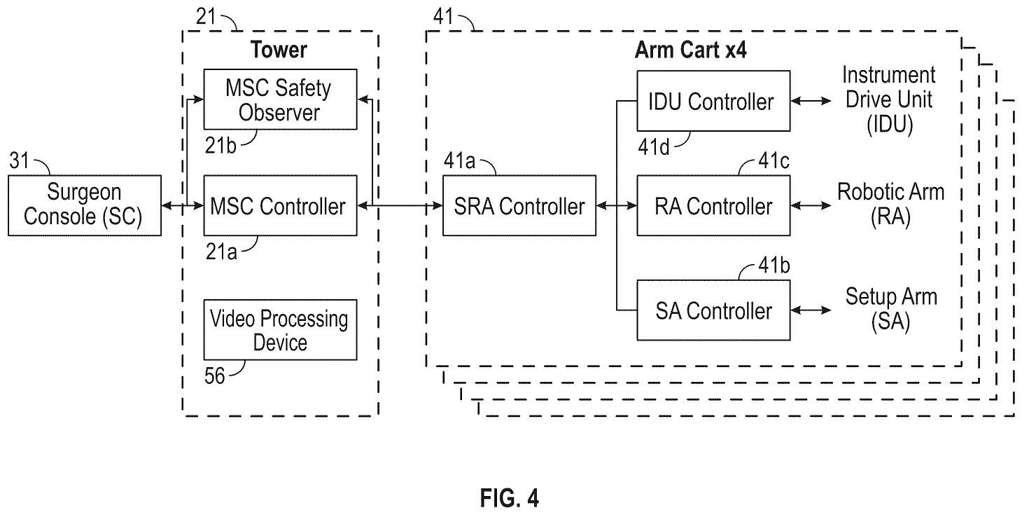

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

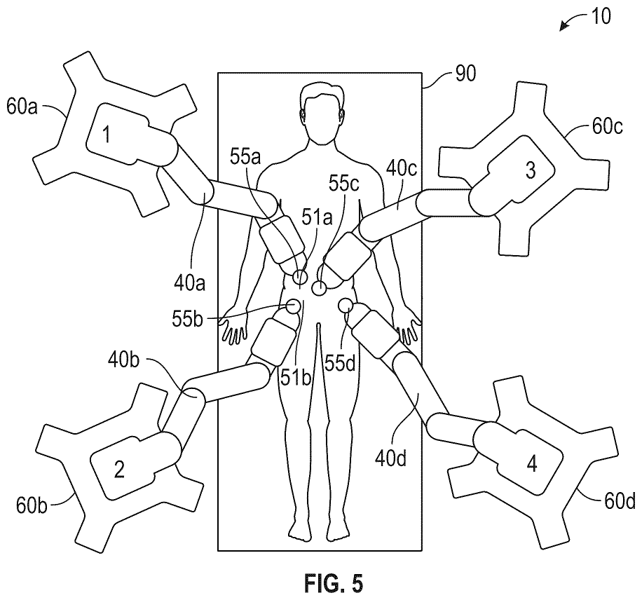

With reference to FIG. 5, the surgical robotic system 10 is set up around a surgical table 90. The surgical robotic system 10 includes movable carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placements are determined, the access ports 55a-d are inserted into the patient, and carts 60a-d are positioned to insert instruments 50 and the endoscopic camera 51 into corresponding ports 55a-d.

During use, each of the robotic arms 40a-d is attached to one of the access ports 55a-d that is inserted into the patient by attaching the port latch 46c (FIG. 2) to the access port 55 (FIG. 3). The IDU 52 is attached to the holder 46, followed by the SIM 43 being attached to a distal portion of the IDU 52. Thereafter, the instrument 50 is attached to the SIM 43. The instrument 50 is then inserted through the access port 55 by moving the IDU 52 along the holder 46.

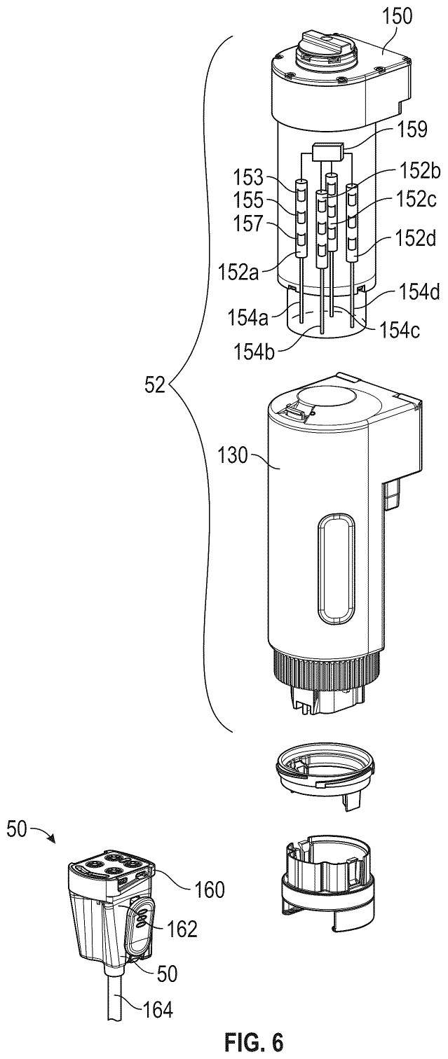

With reference to FIG. 6, the IDU 52 is shown in more detail and is configured to transfer power and actuation forces from its motors 152a-d to the instrument 50 to drive movement of components of the instrument 50, such as articulation, rotation, pitch, yaw, clamping, cutting, etc. The IDU 52 may also be configured for the activation or firing of an electrosurgical energy-based instrument or the like (e.g., cable drives, pulleys, friction wheels, rack and pinion arrangements, etc.).

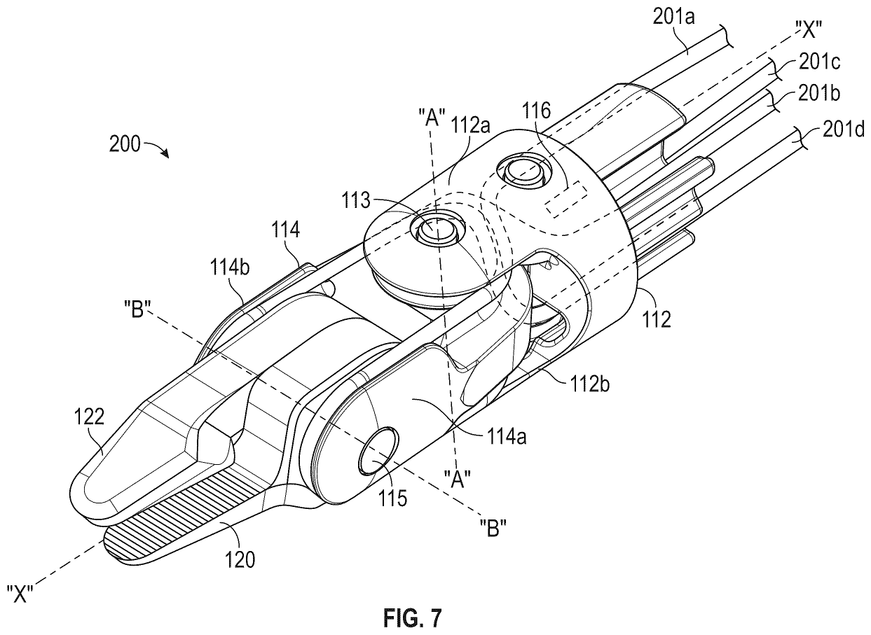

The IDU 52 includes a motor pack 150 and a sterile barrier housing 130. Motor pack 150 includes motors 152a-d for controlling various operations of the instrument 50. The instrument 50 is removably couplable to IDU 52. As the motors 152a-d of the motor pack 150 are actuated, rotation of the drive transfer shafts 154a, 154b, 154c, 154d of the motors 152a-d, respectively, is transferred to drive assemblies of the instrument 50. The instrument 50 is configured to transfer rotational forces/movement supplied by the IDU 52 (e.g., via the motors 152a-d of the motor pack 150) into longitudinal movement or translation of the cables or drive shafts to effect various functions of an end effector 200 (FIG. 7).

Each of the motors 152a-d includes a current sensor 153, a torque sensor 155, and a position sensor 157, which may be an angular motor position sensor. For conciseness only, operation of the motor 152a is described below. The sensors 153, 155, 157 monitor performance of the motor 152a. The current sensor 153 is configured to measure current draw of the motor 152a and the torque sensor 155 is configured to measure motor torque. The torque sensor 155 may be any force or strain sensor including one or more strain gauges configured to convert mechanical forces and/or strain into a sensor signal indicative of the torque output by motor 152a. Position sensor 157 may be any device that provides a sensor signal indicative of the number of rotations of the motor 152a, such as a mechanical encoder or an optical encoder. Parameters which are measured and/or determined by position sensor 157 may include speed, distance, revolutions per minute, position, and the like. Sensor signals from sensors 153, 155, 157 are transmitted to the IDU controller 41d (FIG. 4), which then controls the motors 152a-d based on the sensor signals, via an actuator controller 159. In particular, the actuator controller 159 controls torque outputted and angular velocity of the motors 152a-d. In embodiments, additional position sensors may also be used, which include, but are not limited to, potentiometers coupled to movable components and configured to detect travel distances, Hall Effect sensors, accelerometers, and gyroscopes. In embodiments, a single controller can perform the functionality of the IDU controller 41d and the actuator controller 159.

With continued reference to FIGS. 6 and 7, instrument 50 includes an adapter 160 having a housing 162 at a proximal end portion thereof and an elongated shaft 164 that extends distally from housing 162. The instrument 50 also includes the end effector 200 as shown in FIG. 7. The housing 162 is configured to selectively couple to IDU 52, to enable the motors 152a-d of IDU 52 to operate the end effector 200 of the instrument 50 (FIG. 7). As shown in FIG. 6, the housing 162 supports a drive assembly (not shown) that mechanically and/or electrically cooperates with the motors 152a-d of the IDU 52. The drive assembly of instrument 50 may include any suitable electrical and/or mechanical component to effectuate driving force/movement.

The instrument 50 also includes an end effector 200 coupled to the elongated shaft 164. The end effector 200 may include any number of degrees of freedom allowing the end effector 200 to articulate, pivot, etc., relative to the elongated shaft 164. The end effector 200 may be any suitable surgical end effector configured to treat tissue, such as a dissector, grasper, sealer, stapler, etc.

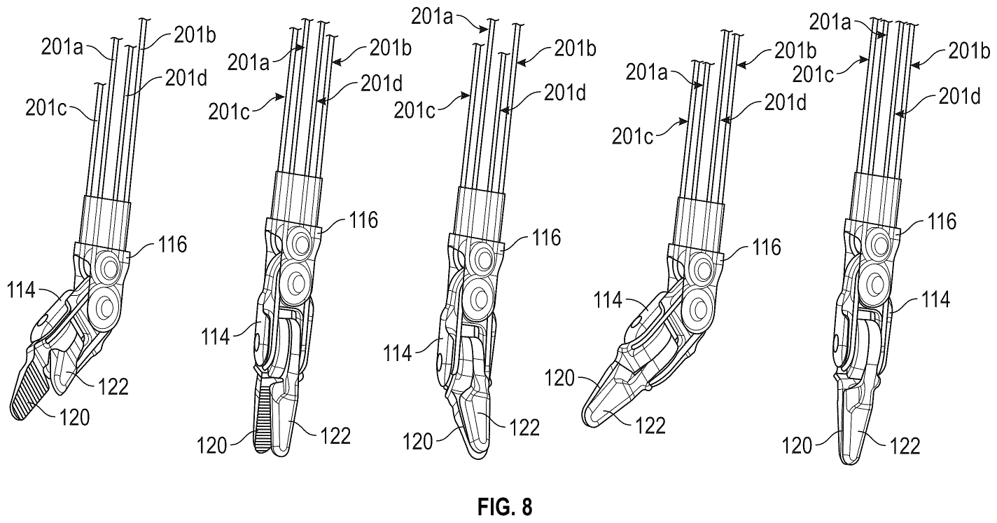

As shown in FIGS. 7 and 8, the end effector 200 may include a pair of opposing jaws 120 and 122 that are movable relative to each other. The jaws 120 and 122 may be grippers as shown or any other suitable type of jaws, e.g., shears, sealers, etc. In embodiments, the end effector 200 may include a proximal portion 112 having a first axle 113 and a distal portion 114 (i.e., wrist portion). The end effector 200 may be actuated using a plurality of cables 201a-d routed through proximal and distal portions 112 and 114 around their respective pulleys 112a, 112b, 114a, 114b, which are integrally formed as arms of the proximal and distal portions 112 and 114. Each of the cables 201a-d is actuated by a respective motor 152a-d via corresponding couplers disposed in adapter 160. In embodiments, the end effector 200, namely, the distal portion 114 and the jaws 120 and 122, may be articulated about the axis “A-A” to control a yaw angle of the end effector with respect to a longitudinal axis “X-X”. The distal portion 114 includes a second axle 115 with first jaw 120 and a second jaw 122 pivotably coupled to the second axle 115. The jaws 120 and 122 are configured to pivot about an axis “B-B” defined by the second axle 115 allowing for controlling a pitch angle of the jaws 120 and 122 as well as opening and closing the jaws 120 and 122. The yaw, pitch, and jaw angles between the jaws 120 and 122 as they are moved between open and closed positions are controlled by adjusting the tension and/or length and direction (e.g., proximal or distal) of the cables 201a-d as shown in FIG. 8. The end effector 200 may also include a cable displacement sensor 116 configured to measure position of the cables 201a-d. Thus, the end effector 200 may have three degrees of freedom, yaw, pitch, and jaw angle between jaws 120 and 122. Control algorithms for a cable actuated instrument 50 are also described in International Patent Application No. PCT/US2022/019703, “Surgical Robotic System for Realignment of Wristed Instruments,” filed on Mar. 10, 2022.

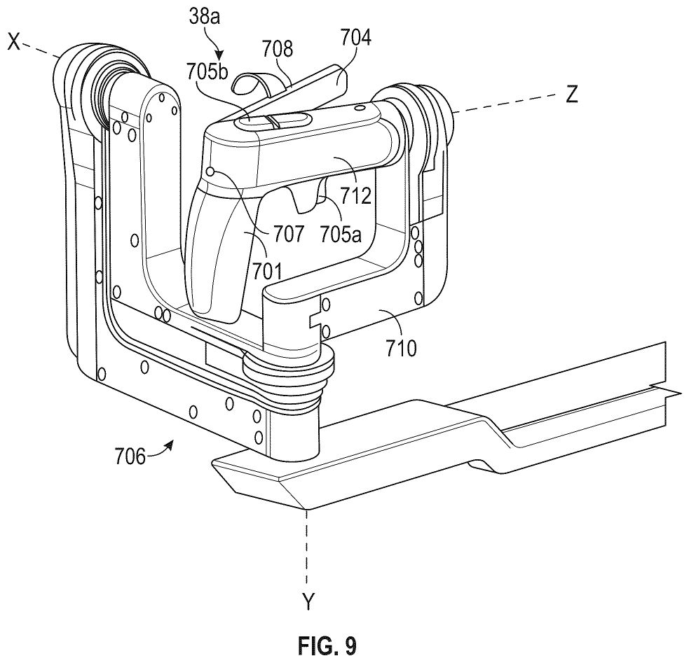

FIG. 9 shows the left-handle controller 38a, which is a mirror copy of the right-handle controller 38b. Each of the handle controllers 38a and 38b includes a handle 701 and a paddle 708 that is pivotally coupled to the handle 701 at one end (e.g., proximal) of the paddle 708. The paddle 708 is configured to control actuation, namely, opening and closing jaws 120 and 122 of the end effector 200. Operation of the paddle 708 and the operation is described with respect to the end effector 200 but may apply to other end effector as well. The paddle 708 may include a finger sensor 704 configured to detect presence or movement of a finger, such as touch sensors, capacitive sensors, optical sensors, and the like. In embodiments, the finger sensor 704 may be disposed on any portion of the handle controllers 38a and 38b. Each of the handle controllers 38a and 38b may also include a trigger 705a and one or more buttons 705b for activating various functions of the instrument 50. In addition, each of the handle controllers 38a and 38b may include a gimbal assembly 706 allowing for movement and rotation of the handle controllers 38a and 38b about three axes (x, y, z). The handle controllers 38a and 38b may also include an infrared proximity sensor 707 configured to detect hand contact with a grip of the handle controllers 38a and 38b. The controller 31a of the surgeon console 30 monitors operator interactions with the handle controllers 38a and 38b and controls the instrument(s) 50 in response to operator inputs.

The paddle 708 is maintained, i.e., biased, in an open position by a motor 712 (e.g., a feedback motor), which receives operator mechanical input, i.e., as the motor 712 is back driven during movement of the paddle 708 in the opposite direction, e.g., if the paddle 708 is being moved to close, the motor 712 drives to open and vice versa. The paddle 708 may include a finger catch to allow for movement of the paddle 708 away from the handle 701.

The motor 712 also provides force feedback to the paddle 708 by counteracting operator's input, i.e., the motor 712 is forward driven. In addition, the motor 712 also measures the force, angle relative to the handle 701, and/or velocity of the paddle 708. The angle of the paddle 708 relative to the handle 701 is proportional to the angle between jaws 120 and 122. Thus, the paddle 708 and the jaws 120 and 122 may be fully aligned when in fully open and fully closed position and the jaw angle in between those position corresponds the paddle angle during the travel of the paddle 708.

In addition, the controller 31a also monitors individual or a new velocity of each joint of the gimbal assembly 706 as well as displacement of each of the joint of the gimbal assembly 706 and/or net displacement of the gimbal assembly 706. Details of the handle controllers 38a and 38b are provided in U.S. Patent Application Publication No. 2020/0315729, titled “Control arm assemblies for robotic surgical systems”.

A haptic feedback device 710 is disposed in the handle controller 38b to provide vibratory or haptic feedback to the operator. As shown, the haptic feedback device 710 is configured to provide vibrational feedback at set frequencies and intervals to provide a sensation of touching. The haptic feedback device 710 may include eccentric rotating mass (ERM) actuator, a linear resonant actuator (LRA), a piezoelectric actuator, or any other suitable tactile actuator configured to impart information to the operator through their sense of touch. Details of the haptic feedback mechanism are provided in U.S. Pat. No. 10,517,686, titled “Haptic feedback controls for a robotic surgical system interface”.

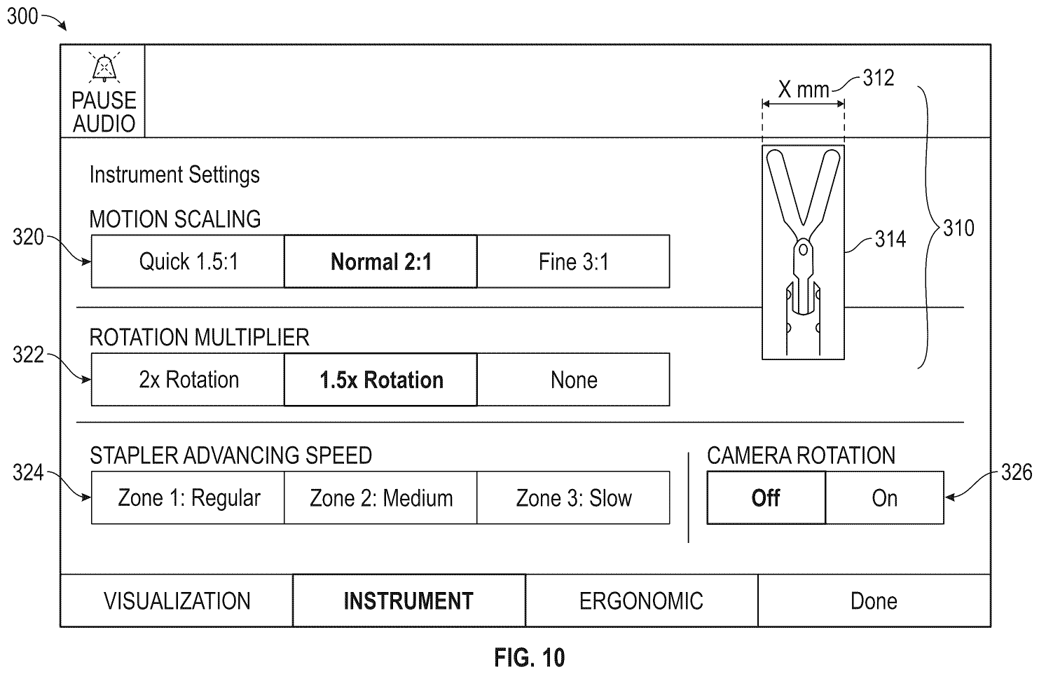

FIG. 10 shows an exemplary graphical user interface (GUI) 300 for user-controlled paddle mapping. The GUI 300 may be displayed on any of the display screens 23, 32, or 34. The GUI may include various user interface elements for adjusting instrument settings relating to an instrument, such as instrument 50, including an aperture interface element 310, a motion scaling interface element 320, a rotation multiplier interface element 322, a stapler advancement interface element 324, and a camera rotation interface element 326.

The aperture interface element 310 may enable a clinician to select a maximum aperture size 312 (e.g., a maximum aperture opening size) for a corresponding end effector 200 of any of the instruments 50 being used by the system 10. For example, a clinician may select a maximum aperture size 312 corresponding to an opening defined between jaws 120, 122 of an end effector 200. However, it will be understood that various end effectors (e.g., dissector, sealer, stapler, etc.) may be compatible with the paddle mapping feature.

The aperture interface element 310 may include an interactive icon 314. The interactive icon 314 may be a user-adjustable graphical representation of an end effector, such as end effector 200 with jaws 120, 122, as shown in FIG. 10. For example, GUI 300 may include a touch screen enabling a clinician to drag portions of interactive icon 314 (e.g., portions representing first and/or second jaws 120, 122) in a proximal and/or distal direction to set the maximum aperture size 312. A numerical indication of the maximum aperture size may be reflected above the interactive icon (e.g., 10 mm). In aspects, GUI 300 may be mapped to an alternative user interface device, such as a dial, keyboard, mouse, or similar device, which may be used to set aperture interface element 310.

In aspects, interactive icon 314 may be a dropdown menu, an alphanumeric input box, a slider, a button, and/or any combination thereof. In aspects, the maximum aperture size 312 range presented and/or selection made may vary based on the clinician's task. For example, when performing fine blunt dissection, a clinician may not want the jaw to completely open, thereby scaling down the open position.

To enable user-controlled paddle mapping, a user interface device such as handle controller 38a or 38b may be mapped to an end effector position. For example, paddle 708 of handle controller 38a or 38b may have an open position mapped to a jaw open position of jaws 120, 122 and/or a closed position mapped to a jaw closed position of jaws 120, 122. The jaw open position and/or the jaw closed position may be scaled between 0 and 1, where 0 is the fully closed position and 1 is the fully open position. The jaw open position may be limited by maximum aperture size 312. For example, if jaws 120, 122 are capable of a maximum open position of 10 mm, but the maximum aperture size is set to 5 mm, then the new scaled open position of jaws 120, 122 will become 5 mm. A clinician may dynamically adjust the maximum aperture size 312 before, during, and/or after a procedure.

It will be understood that various user interface devices may be compatible with GUI 300. For example, trigger 705a and/or one or more buttons 705b of handle controllers 38a and 38b may be mapped to an open position of jaws 120, 122. In another example, handle 701 and/or foot pedal 36 may be mapped to an open position of jaws 120, 122. In aspects, multiple user interface devices may be simultaneously mapped to jaws 120, 122.

With continued reference to FIG. 10, additional instrument settings also may be set with the maximum aperture size 312, such as the motion scaling interface element 320, the rotation multiplier interface element 322, the stapler advancement interface element 324, and/or the camera rotation interface element 326.

Motion scaling interface element 320 may be set to enable motion scaling for paddle 708. Motion scaling interface element 320 may include pre-programmed settings such as Quick (1.5:1), Normal (2:1), and Fine (3:1). The motion scaling may scale the movement of paddle 708 by based on the user-provided ratio, thereby increasing or decreasing the scaled motion of end effector 200 (e.g., the open and/or closed positions of jaws 120, 122). For example, when a 2:1 ratio is set, depression of paddle 708 by 10 mm may equate to a 5 mm movement of end effector 200. Rotation multiplier interface element 322 may be set to adjust a rotation speed of an end effector 200, such as 2× Rotation, 1.5× rotation, and No Rotation. Stapler advancement interface element 324 may set an advancement speed of end effector 200, such as Zone 1 (Regular), Zone 2 (Medium), and Zone 3 (Slow). Camera rotation interface element 326 may rotate a camera lens by a preset angle relative to a plane on the surgical field (e.g., 180 degrees).

In aspects, various default settings may be loaded based on a clinician's profile and/or a type of surgical procedure selected. For example, a clinician may have a preferred default motion scaling and rotation multiplier when operating on thoracic lung tissue. In aspects, certain settings may be locked and therefore not adjustable based on prior setting selections and/or the clinician's profile. For example, as shown in FIG. 10, the stapler advancing speed may be grayed out, i.e., nonadjustable when the system operates in a cutting mode where a stapler is not in use.

As an example, during a surgical procedure, a clinician may select an “Instrument” tab at the bottom of the GUI 300, which will open the “Instrument Settings” menu. There, the clinician may enact various setting selections based on the type of procedure to be performed. For example, during lung resection a clinician may desire fine, blunt dissection to carefully separate tissue. The clinician may navigate to the aperture interface element 310 and pinch the “jaws” of interactive icon 314, thereby decreasing the maximum aperture size of jaws 120, 122 when set to the open position. Therefore, when the clinician provides input via paddle 708 corresponding to the open position of jaws 120, 122, the open position of jaws 120, 122 will be limited to the size of maximum aperture size 312.

After setting the maximum aperture size 312, the clinician may set additional settings, which may be mapped to paddle 708 and/or additional, user-controlled devices. For example, the clinician may set a “Fine 3:1” setting for motion scaling interface element 320, a rotation multiplier a speed of “Zone 3: Slow” for stapler advancement interface element 324, and a camera rotation of 180 degrees for camera rotation interface element 326.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
an instrument including an end effector having a first jaw and a second jaw, wherein at least one of the first jaw or the second jaw is movable relative to the other of the first jaw or the second jaw between a closed configuration and an open configuration; and
a surgeon console including:
an input controller for receiving a first input corresponding to the closed configuration of the first and second jaws and a second input corresponding to the open configuration of the first and second jaws; and
a display screen for displaying a graphical user interface for receiving a user input for setting a maximum opening aperture for the open configuration of the first and second jaws.

2. The surgical robotic system according to claim 1, further comprising an instrument drive unit coupled to the instrument.

3. The surgical robotic system according to claim 2, wherein the instrument drive unit includes a controller and at least one motor for moving at least one of the first jaw or the second jaw between the closed configuration and the open configuration.

4. The surgical robotic system according to claim 3, wherein the controller opens at least one of the first jaw or the second jaw up to the maximum opening aperture.

5. The surgical robotic system according to claim 1, wherein the graphical user interface includes a user interface element for setting the maximum opening aperture.

6. The surgical robotic system according to claim 5, wherein the user interface element is a user-adjustable graphical representation of the instrument.

7. The surgical robotic system according to claim 5, wherein the user interface element is selected from a group consisting of a dropdown menu, an alphanumeric input box, a slider, and combinations thereof.

8. The surgical robotic system according to claim 5, wherein the input controller is selected from a group consisting of a handle controller and a foot pedal.

9. The surgical robotic system according to claim 1, wherein the display screen includes a touch panel for receiving the user input.

10. The surgical robotic system according to claim 1, wherein the input controller includes:
a handle; and
a paddle pivotably movable relative to the handle from a closed position corresponding to the closed configuration of the first and second jaws and an open position corresponding to the open configuration of the first and second jaws.

11. The surgical robotic system according to claim 10, wherein the input controller further includes a trigger moveable relative to the handle from a deactivated position corresponding to the closed configuration of the first and second jaws and an activated position corresponding to the open configuration of the first and second jaws.

12. The surgical robotic system according to claim 10, wherein the input controller further includes a haptic feedback device for generating haptic feedback when the paddle is in at least one of the closed position or the open position.
